(19) United States
(12) Patent Application Publication
(10) Pub. No.: US 2017/0368109 A1
(43) Pub. Date: Dec. 28, 2017

(54) Probiotic Fermentation Products

(71) Applicant: Animal Cell Therapies Inc., San Diego, CA (US)

(72) Inventors: Kathryn J. Petrucci, La Jolla, CA (US); Sarah E. Royer Köster, Pacifica, CA (US)

(21) Appl. No.: 15/636,002

(22) Filed: Jun. 28, 2017

Related U.S. Application Data
(60) Provisional application No. 62/355,420, filed on Jun. 28, 2016.

(51) Int. Cl. A61K 35/742; A61K 9/00

(52) U.S. Cl. CPC A61K 35/742; A61K 9/0046

(57) ABSTRACT

This document relates to materials and methods for treating infections (e.g., ear infections and/or skin infections). For example, methods and materials for using a probiotic fermentation product to treat a veterinary animal (e.g., non-human mammal) having an infection and/or a complication associated with an infection (e.g., hearing loss caused by an ear infection) are provided.

### CROSS-REFERENCE TO RELATED APPLICATIONS

This application claims the benefit of U.S. Provisional Application Ser. No. 62/355,420, filed Jun. 28, 2016. The disclosure of the prior application is considered part of (and is incorporated by reference in) the disclosure of this application.

### BACKGROUND

### 1. Technical Field

This document relates to materials and methods for treating infections (e.g., ear infections and/or skin infections). For example, this document provides methods and materials for using a probiotic fermentation product to treat a veterinary animal (e.g., a dog or cat) having an infection and/or a complication associated with an infection (e.g., hearing loss caused by an ear infection). For example, this document provides methods and materials for using a probiotic fermentation product to decrease the number of pathogenic microbes present at an infection site (e.g., ear and/or skin).

### 2. Background Information

Ear infections in veterinary animals (e.g., dogs and cats) are common, and most veterinary animals suffer from this painful condition sometime during their life.

### SUMMARY

This document provides materials and methods for treating infections (e.g., ear infections and/or skin infections) and/or a complication associated with an infection (e.g., hearing loss caused by an ear infection). For example, this document provides methods and materials for topically administering a probiotic fermentation product to a veterinary animal (e.g., a dog or cat) having an infection and/or a complication associated with an infection (e.g., hearing loss caused by an ear infection) under conditions wherein the severity of the infection is reduced. For example, this document provides methods and materials for topically administering a probiotic fermentation product to a veterinary animal (e.g., a dog or cat) having an infection and/or a complication associated with an infection (e.g., hearing loss caused by an ear infection) under conditions wherein the number of pathogenic microbes present at an infection site (e.g., ear and/or skin) is reduced and/or eliminated. A probiotic fermentation product can be present in a veterinary grooming composition (e.g., ear washes, shampoos, sprays, and pet wipes).

As demonstrated herein, probiotic fermentation products can be effective to treat ear infections. In some cases, a probiotic fermentation product can be used to reduce the symptoms of an infection and/or a complication associated with an infection.

In general, one aspect of this document features a veterinary grooming composition including a probiotic fermentation product. The veterinary grooming composition can be an ear wash, a shampoo, or a pet wipe. The probiotic fermentation product can be a supernatant. The supernatant can be from a species of Bacillus (e.g., Bacillus coagulans). The veterinary can be for a dog or a cat.

In another aspect, this document features a method for treating an infection in a veterinary animal. The method includes, or consists essentially of, identifying said veterinary animal as having said infection, and administering a probiotic fermentation product to said veterinary animal under conditions wherein the severity of one or more symptoms of said infection is reduced. The veterinary animal can be a dog or a cat. The infection can be an ear infection having symptoms of odor, redness, exudate, inflammation, and/or itching. The infection can be a skin infection having symptoms of itchiness, papules, pustules, scales, redness, odor, collarets, flakes, exudate, and/or increased oil production. The probiotic fermentation product can be a supernatant. The supernatant can be from a species of Bacillus (e.g., Bacillus coagulans).

In another aspect, this document features a method for treating an infection in a veterinary animal. The method includes, or consists essentially of, identifying said veterinary animal as having said infection, and administering a probiotic fermentation product to said veterinary animal under conditions where the number of pathogenic microbes present at the infection site is decreased. The veterinary animal can be a dog or a cat. The pathogenic microbes can be species in the genera Escherichia, Staphylococcus, Pseudomonas, Enterococcus, Serratia, Proteus, Klebsiella, Streptococcus, Bacillus, Cryptococcus, Candida, Malassezia, and Aspergillus. The pathogenic microbes present at the infection site can be decreased by at least about 50 percent. The probiotic fermentation product can be a supernatant. The supernatant can be from a species of Bacillus (e.g., Bacillus coagulans).

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A and 1B show effective treatment of canine ear infections using a probiotic fermentation product. (A) A canine ear infection before treatment. (B) The same canine ear shown in (A) after 1 week of treatment with a veterinary ear wash including a probiotic fermentation product.

### DETAILED DESCRIPTION

This document provides methods and materials for treating veterinary infections. Methods for treating a veterinary infection can include contacting the infection site with a composition including one or more probiotic fermentation products. For example, this document provides methods and materials for using a probiotic fermentation product to treat an infection (e.g., an ear infection and/or a skin infection) and/or a complication associated with an infection (e.g., hearing loss caused by an ear infection). In some cases, a probiotic fermentation product can be used to reduce the symptoms of an infection.

When treating a veterinary infection as described herein, the infection can be in any tissue. In some cases, the infection can be a superficial infection. Infections can occur in many tissues within the body including, without limitation, ears (chronic ear infection), skin (pyoderma). In cases where the infection is an ear infection, the infection can be present in a single ear or in both ears (bilateral). In cases where the infection is an ear infection, the infection can be present in the external ear canal, middle ear, and/or the inner ear. In some embodiments, the veterinary infection treated as described herein can be an ear infection, such as chronic bilateral ear infection.

When treating a complication associated with an infection described herein, the complication associated with an infection can be hearing loss caused by an ear infection. Other complications associated with infections include, without limitation, loss of balance, unusual eye movements, walking in circles, pain, pruitis, irritability, and aggression.

In some cases, the materials and methods provided herein can be used to reduce the symptoms of an infection (e.g., an ear infection and/or a skin infection). Symptoms of veterinary ear infections include, without limitation, odor, redness, itching, swelling, exudate, scratching/rubbing of the ear or area around the ear, head shaking, head tilting, discharge, crusts or scabs on inside of the outer ear, hair loss around the ear, pain, irritability, depression, and aggression. Symptoms of veterinary skin infections include, without limitation, itchiness, scales, redness, odor, flakes, exudate, increased oil production, pustules, crusted skin, lesions, loss of hair (alopecia), papules, scaliness, and collarettes.

In some cases, the materials and methods provided herein can be used to decrease the number of pathogenic microbes present at the infection site (e.g., ear and/or skin). Pathogenic microbes causing veterinary infections include bacteria, fungi, viruses, and parasites. In cases where the pathogenic microbe is a bacterium, the pathogenic microbe can be a gram positive bacterium or a gram negative bacterium. In some cases, pathogenic microbes can be a species in the genera Escherichia, Staphylococcus, Pseudomonas, Enterococcus, Serratia, Proteus, Klebsiella, Streptococcus, Bacillus, Cryptococcus, Candida, Malassezia, and Aspergillus. Examples of pathogenic microbes causing veterinary infections include, Escherichia coli, Coagulase positive staphylococcus, Staphylococcus aureus (e.g., methicillin-resistant S. aureus (MRSA)), Beta hemolytic streptococci, Enterococcus faecium (e.g., vancomycin-resistant enterococci (VRE)), Serratia marcescens, Staphylococcus pseudointermedius, Proteus mirabilis, Staphylococcus pseudintermedius, Klebsiella pneumoniae, microbes having multiple drug resistance (MDR), Staphylococcus epidermidis, Staphylococcus schleiferi, Pseudomonas aeruginosa, Malassezia, and Aspergillus.

Any type of veterinary animal having an infection (or a complication associated with an infection) or at risk for developing an infection (or a complication associated with an infection) can be treated as described herein. A veterinary animal can be a non-human mammal. Non-human mammals that can be treated as described herein include, without limitation, primates such as monkeys, cats, dogs, horses, cows, pigs, sheep, ferrets, guinea pigs, rabbits, hamsters, gerbils, chinchillas, hedgehogs, and rodents. For example, non-human mammals such as cats or dogs having an infection can be treated with a probiotic fermentation product.

Any appropriate method can be used to identify a veterinary animal having an infection (or a complication associated with an infection) or as being at risk for developing an infection (or a complication associated with an infection). In some cases, a veterinary animal can be identified as having an ear infection. For example, smelling for odor, visual analysis (e.g., examining the ear canal and ear drum for redness and/or swelling, for example, with an otoscope), discharge sample analysis (e.g., a sample of ear discharge may be examined to look for the presence of pathogenic microbes), tissue/fluid sample analysis (e.g., urine analysis, blood tests, myringotomy), cytology, culture, and/or imaging techniques (e.g., computed tomography (CT), magnetic resonance imaging (MRI) scans) can be used to identify a dog or other veterinary animal having an ear infection. For example, ear anatomy (e.g., the presence of large pendulous ear flaps, shape of the ear canal, narrow ear canals, hairy ear canals), and/or allergic conditions (e.g., food allergies, allergies to dusts and pollens can be used to identify a dog or other veterinary animal as being at risk for developing an ear infection. In some cases, a veterinary animal can be identified as having a skin infection. For example, visual analysis, tissue/fluid sample analysis (e.g., skin scrapings, skin biopsies), cytology, and/or culture can be used to identify a dog or other veterinary animal having a skin infection. For example, the presence of atopic dermatitis, allergic conditions (e.g., allergic skin conditions), parasitic diseases, and/or food hypersensitivities can be used to identify a dog or other veterinary animal as being at risk for developing a skin infection.

In some cases, identifying a veterinary animal having an infection (or a complication associated with an infection) or as being at risk for developing an infection (or a complication associated with an infection) also can include identifying the pathogenic microbe causing the infection. Any appropriate method can be used to identify the pathogenic microbe causing the infection. For example, visual techniques (e.g., examination under a microscope), genetic techniques (e.g., 16S sequencing), cytology, and/or culture can be used to identify one or more pathogenic microbes causing an infection in a dog or other veterinary animal. In some cases, one or more pathogenic microbes can be cultured (e.g., to increase sample sizes) prior to identification.

Once identified as having an infection (or a complication associated with an infection) or as being at risk for developing an infection (or a complication associated with an infection), the veterinary animal (e.g., a dog or cat) can be administered (e.g., topically administered) a composition including one or more probiotic fermentation products.

A composition including one or more probiotic fermentation products described herein can include any fermentation product from any appropriate probiotic. In some cases, probiotics that produce fermentation products can be from a species in the genera Bacillus, Lactobacillus, Bifidobacteria, Streptococcus, and Enterococcus. Examples of probiotics that can be used produce fermentation products include, without limitation, Bacillus coagulans, Lactobacillus acidophilus, Lactobacillus brevis, Lactobacillus bulgaricus, Lactobacillus plantarum, Lactobacillus rhamnosus, Lactobacillus fermentum, Lactobacillus caucasicus, Lactobacillus helveticus, Lactobacillus lactis, Lactobacillus reuteri, Lactobacillus casei, Bifidobacterium bifidum, Bifidobacterium infanti, and Bifidobacterium longum. In some cases, a probiotic can be B. coagulans (e.g., B. coagulans BC30). Fermentation products can be supernatants and/or cell lysates. In some cases, a probiotic fermentation product can be a B. coagulans BC30 supernatant (e.g., as described, for example, in U.S. Pat. No. 9,192,659). A probiotic fermentation product described herein can be made using any appropriate technique. For example, a probiotic can be cultured under anaerobic conditions to produce a probiotic fermentation product.

A composition including one or more probiotic fermentation products can include any appropriate amount of a probiotic fermentation product described herein. For example, a composition including one or more probiotic fermentation products provided herein can contain from about 0.05 percent to about 100 percent (e.g., about 1 percent to about 95 percent, about 2 percent to about 85 percent, about 3 percent to about 75 percent, about 4 percent to about 60 percent, about 5 percent to about 50 percent, about 1 percent to about 80 percent, about 2 percent to about 50 percent, about 3 percent to about 50 percent, or about 4 percent to about 25 percent), by weight, of a probiotic fermentation product. In some cases, a composition (e.g., a veterinary ear wash) including one or more probiotic fermentation products provided herein can contain about 5 percent to about 10 percent of a probiotic fermentation product. In some cases, a composition (e.g., a veterinary shampoo) including one or more probiotic fermentation products provided herein can contain about 5 percent to about 10 percent of a probiotic fermentation product.

In some cases, a composition including one or more probiotic fermentation products (e.g., one, two, three, four, five, or more probiotic fermentation products) can be administered to a veterinary animal (e.g., a dog or cat) to treat an infection (e.g., an ear infection and/or skin infection) and/or a complication associated with an infection (e.g., hearing loss caused by an ear infection).

In some cases, a composition including one or more probiotic fermentation products can be administered (e.g., topically administered) to a veterinary animal (e.g., a dog or cat) having an infection (e.g., an ear infection and/or skin infection) and/or a complication associated with an infection as a combination therapy with one or more additional agents used to treat infections. For example, a combination therapy used to treat a veterinary animal having an infection can include administering to the veterinary animal (e.g., a dog or cat) a composition including one or more probiotic fermentation products and one or more other agents for infection such as antimicrobials (such as aminoglycosides, beta lactam antibiotics, chloramphenicol, diaminopyrimidines (e.g., trimethoprim), glycopeptides, fluroquinolones, polymixins, lincosamides, macrolides, rifamycins, streptogramins, sulfonamides, and tetracyclines), anti-inflammatories (such as prednisone, glucocoritiods, and coriticosteroids), and/or antifungals.

In some cases, a composition including one or more probiotic fermentation products can be administered (e.g., topically administered) to a veterinary animal (e.g., a dog or cat) having an infection (e.g., an ear infection and/or skin infection) and/or a complication associated with an infection as a combination therapy with one or more additional therapies used to treat infections. For example, a combination therapy used to treat a veterinary animal having an infection can include administering to the veterinary animal (e.g., a dog or cat) a composition including one or more probiotic fermentation products and one or more other therapies for infection such as routine ear cleaning (e.g., professional cleaning), surgical drainage, and/or surgical ear resections.

In embodiments where one or more probiotic fermentation products are used in combination with one or more additional agents to treat infections, the one or more additional agents can be administered at the same time or independently. For example, the composition including one or more probiotic fermentation products can be administered first, and the one or more additional agents administered second, or vice versa. In embodiments where one or more probiotic fermentation products are used in combination with one or more additional therapies to treat infections (e.g., routine ear cleaning and/or surgical drainage), the one or more additional therapies can be performed at the same time or independently of the administration of one or more probiotic fermentation products. For example, the composition including one or more probiotic fermentation products can be administered before, during, or after the one or more additional therapies are performed.

A composition containing one or more probiotic fermentation products can be designed for topical, oral, parenteral (including subcutaneous, intramuscular, intravenous, and intradermal), or inhaled administration. When being administered topically, a composition containing one or more probiotic fermentation products can be formulated into a veterinary grooming product. Examples of veterinary grooming products for topical administration include ear washes, shampoos, sprays, and pet wipes.

A composition (e.g., a veterinary grooming composition) containing one or more probiotic fermentation products also can include one or more additional components. Additional components of a veterinary grooming compositions provided herein can be, for example, solvents, astringents, humectants, antiseptic, surfactants, pH balancers, musks, preservatives, emulsifying agents, emollients, thickeners, medicines (e.g., anti-inflammatories), and fragrances. Examples of additional components that can be incorporated into a veterinary grooming composition containing one or more probiotic fermentation products described herein include, without limitation, alcohol (such as ethanol, 1-propanol, isopropanol, cetyl alcohol, stearyl alcohol, cetostearyl alcohol, oleyl alcohol, and fatty alcohol), water, witch hazel (Hamamelis virginiana) extract, calamine lotion, distilled vinegar, and rubbing alcohol, sugar alcohols (such as glycerin, sorbitol, xylitol, and maltitol), polymeric polyols (such as polydextrose), alpha hydroxy acids (such as lactic acid), sodium lactate, caprylyl glycol, boric acid, hydrogen peroxide, polysorbate 20, sodium C14-16 olefin sulfonate, disodium laureth sulfosuccinate, cocamidopropyl betaine, sodium chloride, synthetic musk such as ethylene brassylate, triethanolamine, sodium hydroxide, methylisothiazolinone, phenoxyethanol, safflower (Carthamus tinctorius) seed oil, laureth-4, glycol stearate, PEG-120 methyl glucose dioleate, hexylene glycol, aloe barbadensis leaf juice extract, lavender, mint, eucalyptus, cucumber, melon, lemon, coconut, and vanilla.

In some cases, an ear wash composition containing one or more probiotic fermentation products (e.g., B. coagulans BC30 supernatant) can include witch hazel extract, alcohol, water, glycerin, boric acid, polysorbate 20, ethylene brassylate, triethanolamine, methylisothiazolinone, and lavender.

In some cases, a dog shampoo containing one or more probiotic fermentation products (e.g., B. coagulans BC30 supernatant) can include water, sodium C14-16 olefin sulfonate, disodium laureth sulfosuccinate, glycerin, cocamidopropyl betaine, sodium chloride, glycol stearate, laureth-4, PEG-120 methyl glucose dioleate, safflower seed oil, aloe barbadensis leaf juice extract, sodium lactate, sodium hydroxide, caprylyl glycol, phenoxyethanol, hexylene glycol, and lavender.

Effective doses can vary depending on the severity of the infection and/or complication associate with the infection, the route of administration, the age and general health condition of the veterinary animal (e.g., a dog or cat), excipient usage, the possibility of co-usage with other therapeutic treatments such as use of other agents, and the judgment of the treating veterinarian.

An effective amount of a probiotic fermentation product can be any amount that reduces the severity of a symptom of a condition being treated (e.g., an infection and/or a complication associated with an infection) and/or to decrease the number of pathogenic microbes present at the infection site (e.g., ear and/or skin) without producing significant toxicity to the veterinary animal (e.g., a dog or cat). For example, an effective amount of a probiotic fermentation product (e.g., B. coagulans BC30 supernatant) can be an amount that decreases the number of pathogenic microbes present at the infection site (e.g., ear and/or skin) by at least about 50 percent (e.g., at least about 60%, at least about 70%, at least about 75%, at least about 80%, at least about 85%, at least about 90%, at least about 95%, at least about 98%, or at least about 99%). In some cases, an effective amount of B. coagulans BC30 supernatant can be an amount that decreases the number of pathogenic microbes present at the infection site by at least about 99%. For example, an effective amount of a probiotic fermentation product (e.g., B. coagulans BC30 supernatant) can be an amount that decreases the number of pathogenic microbes present at the infection site (e.g., ear and/or skin) by a logio reduction factor (LRF) of at least about 0.5 (e.g., at least about 0.8, at least about 1.0, at least about 1.2, at least about 1.5, at least about 1.8, at least about 2.0, at least about 2.5, at least about 3.0, at least about 3.5, at least about 4.0, at least about 4.5, at least about 5.0, at least about 5.5, at least about 6.0, or at least about 6.5). In some cases, an effective amount of B. coagulans BC30 supernatant can be an amount that decreases the number of pathogenic microbes present at the infection site by a LRF of at least about 6.0. The effective amount can remain constant or can be adjusted as a sliding scale or variable dose depending on the veterinary animal's response to treatment. Various factors can influence the actual effective amount used for a particular application. For example, the frequency of administration, duration of treatment, use of multiple treatment agents, route of administration, and severity of the condition (e.g., an infection and/or a complication associated with an infection) may require an increase or decrease in the actual effective amount administered.

The frequency of administration can be any frequency that reduces the severity of a symptom of a condition to be treated (e.g., an infection and/or a complication associated with an infection) without producing significant toxicity to the veterinary animal (e.g., a dog or cat). For example, the frequency of administration can be from about once a week to about three times a day, from about twice a month to about six times a day, or from about twice a week to about once a day. The frequency of administration can remain constant or can be variable during the duration of treatment. A course of treatment with a composition containing one or more probiotic fermentation products can include rest periods. For example, a composition containing probiotic fermentation products can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., an infection and/or a complication associated with an infection) may require an increase or decrease in administration frequency.

An effective duration for administering a composition containing one or more probiotic fermentation products can be any duration that reduces the severity of a symptom of the condition to be treated (e.g., an infection and/or a complication associated with an infection) without producing significant toxicity to the veterinary animal. For example, the effective duration can vary from several days to several weeks, months, or years. In some cases, the effective duration for the treatment of an infection and/or a complication associated with in infection can range in duration from about one month to about ten years. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and the severity of one or more symptoms related to the condition being treated (e.g., an infection and/or a complication associated with an infection) can be monitored. Any appropriate method can be used to determine whether or not the severity of a symptom is reduced. For example, the severity of a symptom of an infection can be assessed using smelling for odor, visual analysis (e.g., examining the ear canal and ear drum for redness and/or swelling, for example, with an otoscope), discharge sample analysis (e.g., a sample of ear discharge may be examined to look for the presence of pathogenic microbes), tissue/fluid sample analysis (e.g., urine analysis, blood tests, myringotomy), cytology, culture, and/or imaging techniques (e.g., computed tomography (CT), magnetic resonance imaging (MRI) scans) at different time points. For example, the severity of a symptom of an infection can be assessed about two weeks to two months after treatment.

The invention will be further described in the following examples, which do not limit the scope of the invention described in the claims.

### EXAMPLES

### Example 1

### Probiotic Fermentation Products in Veterinary Compositions

A veterinary composition in the form of an ear wash was prepared by combining the ingredients listed below.

Ear wash composition: witch Hazel (H. Virginiana) extract, alcohol, water, lactobacillus fermentation product, glycerin, boric acid, polysorbate 20, ethylene brassylate, triethanolamine, methylisothiazolinone, and lavender.

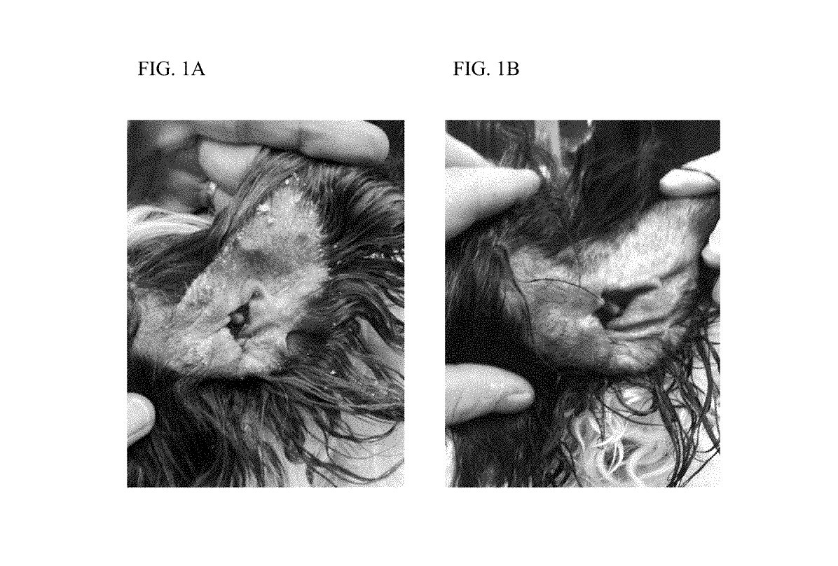

A pre-treatment examination was performed on three dogs having bilateral otitis externa (ear infections). All three dogs presented with severe inflammation through the entire canal and pinna, and demonstrated significant redness, itching, and odor. A representative image of a canine ear infection before treatment is shown in FIG. 1A. Pre-treatment cultures were obtained from one of the dogs (an 11 year old Lhasa Apso mix). The pre-treatment culture from the left ear contained abundant growth of Coagulase positive staphylococcus, Enterococcus sp, Beta hemolytic streptococci, and Staphylococcus pseudointermedius. The pre-treatment culture from the right ear contained abundant growth of Coagulase positive staphylococcus, Enterococcus sp, and Staphylococcus pseudointermedius.

The three dogs were diagnosed as follows:

| Dog | Ailment | Cause | Comments |
|---|---|---|---|
| 1 | bilateral ear | gram positive bacteria | confirming |
| | infection | and exudate | cytology done |
| 2 | bilateral ear | yeast (Malesezzia) | confirming |
| | infection | | cytology done |
| 3 | chronic bilateral | considerable exudate | no cytology |
| | ear infection | and wax | done |

All three dogs were treated by administering the ear wash composition shown above with no other treatment.

After 1 week of treatment, a post-treatment examination was performed. All three dogs exhibited clinically significant improvements in redness, itching and odor. An image of the same canine ear shown before treatment in FIG. 1A is shown after 1 week of treatment with an ear wash including a probiotic fermentation product in FIG. 1B. The two dogs for which confirming cytology was done also improved significantly in the number of organisms seen on cytology. Post-treatment cultures were obtained from the same dog that was cultured pre-treatment. The post-treatment culture from the left ear contained minimal growth of Coagulase positive staphylococcus, Enterococcus sp., Beta hemolytic streptococci, and Staphylococcus pseudointermedius. The post-treatment culture from the right ear contained abundant growth of Coagulase negative staphylococcus, moderate growth of Enterococcus sp., and no growth of Staphylococcus pseudointermedius.

These results demonstrate that formulations including a probiotic fermentation product can be used to treat ear infections effectively in veterinary animals.

### Example 2

### Probiotic Fermentation Products in Veterinary Compositions

A veterinary composition in the form of a shampoo was prepared by combining the ingredients listed below.

Shampoo composition: water, sodium c14-16 olefin sulfonate, disodium laureth sulfosuccinate,glycerin, lactobacillus fermentation product, cocamidopropyl betaine, sodium chloride, glycol stearate, laureth-4, PEG-120 methyl glucose dioleate, Carthamus tinctorius (safflower) seed oil, aloe barbadensis leaf juice extract, sodium lactate, sodium hydroxide, caprylyl glycol, phenoxyethanol, hexylene glycol, and lavender.

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. A veterinary grooming composition comprising a probiotic fermentation product.

2. The veterinary grooming composition of claim 1, wherein said veterinary grooming composition is selected from the group consisting of an ear wash, a shampoo, and a pet wipe.

3. The veterinary grooming composition of claim 1, wherein said probiotic fermentation product is a supernatant.

4. The veterinary grooming composition of claim 3, wherein said supernatant is from a species of Bacillus.

5. The veterinary grooming composition of claim 4, wherein said species of Bacillus is Bacillus coagulans.

6. The veterinary grooming composition of claim 1, wherein said veterinary grooming composition is for a dog or a cat.

7. A method for treating an infection in a veterinary animal, said method comprising:
a) identifying said veterinary animal as having said infection; and
b) administering a probiotic fermentation product to said veterinary animal under conditions wherein the severity of one or more symptoms of said infection is reduced.

8. The method of claim 7, wherein said veterinary animal is a dog or a cat.

9. The method of claim 7, wherein said infection is an ear infection, and wherein said one or more symptoms are selected odor, redness, exudate, inflammation, and itching.

10. The method of claim 7, wherein said infection is a skin infection, and wherein said one or more symptoms are selected from the group consisting of itchiness, papules, pustules, scales, redness, odor, collarets, flakes, exudate, and increased oil production.

11. The method of claim 7, wherein said probiotic fermentation product is a supernatant.

12. The method of claim 11, wherein said supernatant is from a species of Bacillus.

13. The method of claim 12, wherein said species of Bacillus is Bacillus coagulans.

14. A method for treating an infection in a veterinary animal, said method comprising:
a) identifying said veterinary animal as having said infection; and
b) administering a probiotic fermentation product to said veterinary animal under conditions wherein the number of pathogenic microbes present at the infection site is decreased.

15. The method of claim 14, wherein said veterinary animal is a dog or a cat.

16. The method of claim 14, wherein said pathogenic microbes are selected from species in the genera Escherichia, Staphylococcus, Pseudomonas, Enterococcus, Serratia, Proteus, Klebsiella, Streptococcus, Bacillus, Cryptococcus, Candida, Malassezia, and Aspergillus.

17. The method of claim 14, wherein said number of pathogenic microbes present at the infection site are decreased by at least about 50 percent.

18. The method of claim 14, wherein said probiotic fermentation product is a supernatant.

19. The method of claim 18, wherein said supernatant is from a species of Bacillus.

20. The method of claim 19, wherein said species of Bacillus is Bacillus coagulans.
